(19) United States
(12) Patent Application Publication
(10) Pub. No.: US 2009/0246886 A1
(43) Pub. Date: Oct. 1, 2009

(54) LATERAL FLOW TEST STRIP WITH MIGRATING LABEL

(75) Inventor: Robert L. Buck, Fairview, OR (US)

Correspondence Address: KLARQUIST SPARKMAN, LLP, 121 SW SALMON STREET, SUITE 1600, PORTLAND, OR, 97204, US

(73) Assignee: QuantRx Biomedical Corporation

(21) Appl. No.: 12/398,915

(22) Filed: Mar. 5, 2009

Related U.S. Application Data
(63) Continuation of application No. PCT/US2007/019495, filed on Sep. 6, 2007.
(60) Provisional application No. 60/842,816, filed on Sep. 6, 2006.

(51) Int. Cl. G01N 33/566; G01N 31/22

(52) U.S. Cl. 436/501; 422/56

(57) ABSTRACT

A lateral flow test strip is disclosed that includes a path of flow from a sample receiving zone through a mobilization zone to a primary capture zone and a secondary capture zone. A mobilizable conjugate is present in the mobilization zone, and a mobilizable label is present on the test strip upstream of the conjugate. An immobilized first specific binding partner is present in the primary capture zone and an immobilized second specific binding partner is present in the secondary capture zone. The conjugate includes a primary specific binding partner for the first specific binding partner in the primary capture zone, and a secondary specific binding partner that binds the label and the second specific binding partner. Application of liquid sample to the sample receiving zone results in movement of the liquid sample along the path of flow to move the label and conjugate distally along the test device. The label binds the conjugate after the conjugate binds the analyte, the first specific binding partner or second specific binding partner, so that labeling of the conjugate is delayed.

### CROSS REFERENCE TO RELATED APPLICATION

This application is a continuation of copending International Patent Application PCT/US2007/019495, filed Sep. 6, 2007, which claims the benefit of U.S. Provisional Application No. 60/842,816, filed Sep. 6, 2006, both of which are incorporated by reference herein in their entirety.

### FIELD

This disclosure concerns lateral-flow test strips, as well as methods of using them to detect the presence and/or determining an amount of small and large analytes in liquid samples.

### BACKGROUND

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). These assays have also been used for many other purposes, including the analysis of environmental samples (such as natural fluids and industrial plant effluents). Some of the point-of-use assays are based on highly specific interactions between specific binding pairs, such as antigen/antibody, hapten/antibody, lectin/carbohydrate, apoprotein/cofactor and biotin/streptavidin. The assays are often performed with test strips in which a specific binding pair member is attached to a mobilizable material (such as a metal sol or beads made of latex or glass) or an immobile substrate (such as glass fibers, cellulose strips or nitrocellulose membranes). Particular examples of some of these assays are shown in U.S. Pat. Nos. 4,703,017; 4,743,560; and 5,073,484.

Immunochromatographic assays are characterized as either “sandwich” or “competitive” assays. In sandwich assays, a liquid sample that may contain the analyte is mixed with antibodies to the analyte. The antibodies are mobile and typically are linked to a signaling reagent or other label, such as dyed latex, a colloidal metal sol, or a radioisotope. The liquid mixture is then applied to a chromatographic medium (such as a lateral flow test strip) containing a test band or zone of immobilized antibodies that specifically recognize the analyte of interest. When the analyte to be assayed and the labeled antibody reaches the test zone, the immobilized antibodies in the test zone bind the analyte which is in turn bound by the labeled antibodies. The labeled antibodies now immobilized in the test zone provide a visible signal that indicates the analyte is present in the sample. Sandwich assays can be used to obtain either quantitative or semi-quantitative results. Examples of sandwich immunoassays performed on test strips are described in U.S. Pat. Nos. 4,168,146 and 4,366,241.

A competitive immunoassay uses a sample of labeled analyte corresponding to the analyte to be detected or determined, rather than labeled binding partner. The labeled analyte or analyte analogue competes with any unlabeled analyte present in the sample for binding to an antibody in a test zone. The absence of analyte in the sample is indicated by the presence of signal from the labeled analyte/analog that binds to the test zone, and the signal is reduced in proportion to the amount of analyte in the sample that binds to the test zone in competition with the labeled analyte/analog. A drawback to such assays is that a negative signal is often provided, with the presence of analyte corresponding to a weaker signal from the test zone. Examples of competitive immunoassay devices are those disclosed in U.S. Pat. Nos. 4,235,601; 4,442,204; and 5,208,535.

Solid phase immunoassay devices provide a solid support to which one member of a ligand-receptor pair (usually an antibody, antigen, or hapten) is bound. Early forms of solid supports included plates, tubes, or beads of polystyrene, which were known from the fields of radioimmunoassay and enzyme immunoassay. More recently, porous materials such as nylon, nitrocellulose, cellulose acetate, glass fibers, and other porous polymers have been employed as solid supports in the form of test strips.

Some test strip assays in the past took the form of dipsticks, for example home pregnancy and ovulation detection kits, in which antibodies were bound to a solid phase. The test strip was “dipped” into a sample suspected of containing the analyte to mobilize the reagents, and enzyme-labeled antibody was added, either simultaneously or after an incubation period. The test strip was then washed and inserted into a second solution containing a substrate for the enzyme. The enzyme-label, if present, interacted with the substrate, causing the formation of colored products, which either deposited as a precipitate onto the solid phase or produced a visible color change in the substrate solution. EP-A 0 125 118 discloses such a sandwich type dipstick immunoassay. EP-A 0 282 192 discloses a dipstick device for use in competition type assays.

Flow-through type immunoassay devices (also referred to as lateral flow assays) were designed to avoid the need for incubation and washing steps associated with dipstick assays. U.S. Pat. No. 4,632,901 discloses a sandwich immunoassay device wherein antibody (specific to a target antigen analyte) is bound to a porous membrane or filter to which a liquid sample is added. As the liquid flows through the membrane, target analyte binds to the antibody. The addition of sample is followed by addition of labeled antibody. The visual detection of labeled antibody provides an indication of the presence of target antigen analyte in the sample.

Migration assay devices usually incorporate within them reagents that have been attached to colored labels, thereby permitting visible detection of the assay results without addition of further substances. See, for example, U.S. Pat. No. 4,770,853; WO 88/08534; and EP-A 0 299 428.

There are a number of commercially available lateral-flow type tests and patents disclosing methods for the detection of large analytes (MW greater than 1,000 Daltons) as the analyte flows through multiple zones on a test strip. Examples are found in U.S. Pat. No. 5,229,073 (measuring plasma lipoprotein levels), and U.S. Pat. Nos. 5,591,645; 4,168,146; 4,366,241; 4,855,240; 4,861,711; 5,120,643; European Patent No. 0296724; WO 97/06439; and WO 98/36278.

There are also lateral-flow type tests for the detection of small-analytes (MW 100-1,000 Daltons). Generally, these small analyte tests use competitive inhibition to produce negative or indirect reporting results (reduction of signal with increasing analyte concentration), as exemplified by U.S. Pat. No. 4,703,017. Other assays for detecting small analytes using lateral-flow tests that produce positive or direct reporting results (an increase in signal with increasing analyte concentration) are shown in U.S. Pat. Nos. 5,451,504; 5,451,507; 5,798,273; and 6,001,658.

Multiple zone lateral flow test strips are disclosed in U.S. Pat. No. 5,451,504, U.S. Pat. No. 5,451,507, and U.S. Pat. No. 5,798,273.

U.S. Pat. No. 6,656,744 (Pronovost et al.) discloses a lateral flow test strip in which a label binds to an antibody through a streptavidin-biotin interaction.

U.S. Pat. No. 6,001,658 (Fredrickson) discloses a test strip in which a liquid sample flows downstream from a sample application zone through a primary and secondary capture zone. In one example, the sample application zone contains a labeled anti-analyte antibody tracer, the primary capture zone contains immobilized analyte, and the secondary zone contains an antibody that specifically binds the anti-analyte antibody. A liquid sample is applied to the application zone to mobilize the tracer which then binds to any analyte in the sample. If analyte is present in the sample, binding of the antibody tracer to the immobilized analyte in the primary capture zone is inhibited, and increased tracer signal is observed in the secondary capture zone. One problem with this design is that the antibody tracer is a colored particle to which multiple anti-analyte antibodies attach. These multiple binding sites on the tracer decrease the efficiency with which one analyte molecule blocks the antibody-tracer molecule from attaching to the primary capture zone.

U.S. Pat. No. 6,699,722 (Bauer et al.) also discloses a lateral flow test strip in which a liquid sample flows from a sample application zone through a primary and secondary capture zone. In one embodiment, the sample application zone contains a mobilizable tracer made from an analyte analog bound to a colored particle. The primary and secondary capture zones each contain immobilized anti-analyte antibody. A liquid sample is applied to the test strip to mobilize the tracer, but analyte in the sample migrates ahead of the tracer to the primary capture zone where it occupies binding sites of the antibody. The occupied binding sites in the primary capture zone permit the tracer to move through that zone to the secondary capture zone, where the tracer signal indicates the presence of the analyte in the sample. Although the differential migration mechanism is a substantial improvement over the prior art, it is difficult to quantitate the number of antibody binding sites in the primary capture zone. If the capture zone contains too many antibody active sites then it becomes difficult to measure very small quantities of analyte. The number of active sites can be lowered by reducing the number of antibodies immobilized in the primary capture zone, but at the risk of reducing the ability of the primary capture zone to efficiently capture the analyte.

It would be advantageous to provide a lateral flow test strip assay, such as a competitive assay, that avoids the problems of prior art test strips wherein an antibody or analyte is complexed to a visually detectable label such as a colored particle.

### SUMMARY

Disclosed is an embodiment of an improved lateral flow test strip that is capable of detecting an analyte in a liquid sample, and can also determine an amount of the analyte in the sample. The test strip is a bibulous matrix that defines a liquid flow path from a sample receiving zone through a mobilization zone to a primary capture zone and a secondary capture zone. A mobilizable detectable label is present upstream of the mobilization zone, for example in the sample receiving zone, and a mobilizable conjugate is present in the mobilization zone. The primary capture zone contains an immobilized first specific binding partner, which may be the analyte, a binder for the analyte, or an analog (including a fragment) of the analyte. The secondary capture zone contains an immobilized second specific binding partner. The conjugate includes a primary specific binding partner and a secondary specific binding partner. The primary specific binding partner in the conjugate binds the first specific binding partner in the primary capture zone, and the secondary specific binding partner in the conjugate binds the label and the second specific binding partner in the secondary capture zone.

When liquid sample is applied to the sample receiving zone, the liquid moves along the liquid flow path to move the label and conjugate distally along the test device, and the label binds the conjugate after the conjugate binds the analyte, the first specific binding partner or the second specific binding partner. In some embodiments, the label binds the conjugate after the conjugate binds the first specific binding partner or the second specific binding partner. The delayed labeling of the conjugate provides improved lateral flow assays that avoid, for example, the drawback of using colored particle labels that carry large numbers of antibodies. Separation of the wave fronts that carry the label and the conjugate, for example by delaying the rate of flow of the label relative to the conjugate, can permit the conjugate to reach the primary capture zone and interact with it before the label reaches the primary capture zone.

In some disclosed embodiments, the first specific binding partner in the primary capture zone is the analyte or an analog of the analyte, and the primary specific binding partner in the conjugate is an antibody that specifically binds the analyte or the analog. Since the antibody is not carried in high valence (at least 50 binding sites per particle, such as 50-200 binding sites per particle) by the detectable tracer (such as a colored particle), the conjugate has a low valence for binding to the analyte or analog in the primary capture zone. This allows for greater sensitivity, because fewer analyte molecules are required to achieve competitive displacement. For example the ratio of primary specific binding partner to secondary specific binding partner in the conjugate may be less than about 3:1, for example 2:1 or even 1:1. This low ratio permits the conjugate to bind to the analyte or analog target in the primary capture zone in a low ratio of conjugate to target. For example, the conjugate:target ratio can be less than about 4:1, 3:1, 2:1, for example about 1:1.

The application of liquid sample to the test strip moves the label and conjugate toward the primary and secondary capture zones, preferably with the label migrating behind the conjugate. If analyte is present in the sample, the analyte binds to the antibody portion of the conjugate (the primary specific binding partner) such that binding of the antibody portion of the conjugate to analyte or analog in the primary capture zone is inhibited. The conjugate and bound analyte therefore tend to move through the primary capture zone to the secondary capture zone where the secondary specific binding partner binds to the second binding partner. The conjugate is made visually detectable by the label that has also migrated to the secondary capture zone and bound to the secondary specific binding partner portion of the conjugate. If the analyte is absent from the sample, the antibody in the conjugate binds at the primary capture zone, and the conjugate in the primary capture zone is made visually detectable by subsequent binding of the label to the conjugate.

In other disclosed embodiments, the first specific binding partner in the primary capture zone is an antibody that binds the analyte or analog of the analyte. The primary specific binding partner in the conjugate is the analyte or an analog of the analyte. Application of liquid sample to the test strip moves the label and conjugate toward the primary and secondary capture zones, preferably with the label migrating behind the conjugate. If analyte is present in the sample, the analyte binds to the antibody in the primary capture zone to occupy those sites and inhibit binding of the conjugate to the primary capture zone; hence the conjugate moves through the primary capture zone to the secondary capture zone where the secondary specific binding partner in the conjugate binds to the second specific binding partner in the secondary capture zone. If analyte is absent from the sample, the analyte or analog in the conjugate binds to the antibody in the primary capture zone. The conjugate is then made visually detectable by the label that has also migrated along the strip and binds to the secondary specific binding partner in the conjugate.

In some disclosed embodiments the label includes a moiety identical to the second specific binding partner. For example, the second specific binding partner in the secondary capture zone may be streptavidin, and the label may be streptavidin conjugated to a detectable moiety, such as colloidal gold, a fluorescent compound or a colored latex particle.

It is believed to be particularly advantageous for the label to migrate in the liquid sample behind the conjugate, so that the conjugate can bind at either the primary or secondary capture zone before the conjugate is labeled. For example, the conjugate can flow along the liquid flow path in a first wavefront in advance of a second wavefront in which the label flows, at least until the first wavefront reaches the primary capture zone and the conjugate interacts with the first specific binding partner. Alternatively the second wavefront does not overtake the first wavefront until the conjugate reaches the secondary capture zone. Migration of the label along the bibulous matrix in the second wavefront is retarded relative to the first wavefront, for example, by one or more of a combination of label size, label weight, label location and selective retardation of release of label from the matrix. In some examples the label is separated from the conjugate on the bibulous matrix by a sufficient distance that the second wavefront that contains the label migrates behind and does not overtake the first wavefront until the conjugate has bound at the primary or secondary capture areas.

Methods are also disclosed for detecting an analyte in a liquid sample by applying the liquid sample to the sample receiving zone of the test device, so that the liquid transports the detectable label and the conjugate to the primary capture zone and the secondary capture zone. The detectable label migrates behind the conjugate to the primary and secondary capture zones, to label the conjugate after it has bound in either the primary or secondary capture zone.

The foregoing and other features and advantages will become more apparent from the following detailed description of several embodiments, which proceeds with reference to the accompanying figures.

### BRIEF DESCRIPTION OF THE FIGURES

FIG. 1 is a top perspective view of one embodiment of a lateral flow test strip in accordance with the disclosed examples, with the shaded regions representing the sample receiving zone, mobilization zone, and the primary and secondary capture zones.

FIG. 2 is a side view of the test strip shown in FIG. 1, but also illustrating the location on the test strip of the label L, conjugate 1° SBP-2° SBP, first specific binding partner SBP1, and second specific binding partner SBP2. Immobilization of SBP1 and SBP2 is indicated by a line connecting them to the test strip, while label L and conjugate 1° SBP-2° SBP are mobilizable.

FIG. 3 is a top view of the test strip of FIG. 1, but schematically illustrating a competitive assay.

FIG. 4 is a top view of the test strip of FIG. 1, but schematically illustrating a sandwich assay.

FIG. 5 illustrates a lateral flow assay with a migrating label, in which FIG. 5A shows the initial position of the reagents before liquid sample is applied, and FIGS. 5B-5D show a progressively advancing liquid front in which the conjugate (1° SBP-2° SBP) and label (L) advance toward (FIG. 5B) and through the first capture zone (FIG. 5C) and secondary capture zone (FIG. 5D).

FIG. 6 is a schematic drawing that illustrates the operation of an example of a competitive lateral flow assay with a migrating antibody label, with the results obtained in the presence and absence of analyte (the sample-derived analyte indicated by \textcircled{A}).

FIG. 7 is a schematic drawing that illustrates the operation of an example of a competitive assay with a migrating analyte label, with the results obtained in the presence and absence of analyte (the sample-derived analyte indicated by \textcircled{A}).

### DETAILED DESCRIPTION

### I. Abbreviations

A: analyte or analyte analog
\textcircled{A}: sample-derived analyte
A-B: analyte or analyte analog bound to biotin
Ab-B antibody-biotin
B: biotin
L: label
Mob: mobilization zone
SA: streptavidin
*SA: streptavidin with an attached indicator that makes it detectable
SBP: specific binding pair
SBP1: first specific binding partner 1
SBP2: second specific binding partner 2
1° SPB: primary specific binding partner
2° SBP: secondary specific binding partner

### II. Terms and Techniques

Unless otherwise noted, technical terms are used according to conventional usage. Definitions of common terms in molecular biology may be found in The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided, along with the context of some of the terms in the present disclosure:

Analyte: an atom, molecule, group of molecules or compound of natural or synthetic origin (such as, but not limited to, a drug, hormone, enzyme, growth factor antigen, antibody, hapten, lectin, apoprotein, polypeptide, cofactor) sought to be detected or measured that is capable of binding specifically to at least one binding partner (such as, but not limited to, a drug, hormone, antigen, antibody, hapten, lectin, apoprotein, cofactor).

The devices and methods disclosed herein can be practiced with assays for virtually any analyte. The analytes may include, but are not limited to, antibodies to infectious agents (such as HIV, HTLV, Helicobacter pylori, hepatitis, measles, mumps, or rubella), cocaine, benzoylecgonine, benzodiazepine, tetrahydrocannabinol, nicotine, ethanol theophylline, phenytoin, acetaminophen, lithium, diazepam, nortriptyline, secobarbital, phenobarbitol, methamphetamine, theophylline, testosterone, estradiol, estriol, 17-hydroxyprogesterone, progesterone, thyroxine, thyroid stimulating hormone (TSH), follicle stimulating hormone (FSH), luteinizing hormone (LH), transforming growth factor alpha, epidermal growth factor (EGF), insulin-like growth factor (ILGF) I and II, growth hormone release inhibiting factor, IGA and sex hormone binding globulin; and other analytes including antibiotics (such as penicillin), glucose, cholesterol, caffeine, cotinine, corticosteroid binding globulin, PSA, or DHEA binding glycoprotein.

Analytes to be detected by the disclosed assays vary in size. Merely by way of example, small molecule analytes can be, for instance, <0.1 nm (such as cotinine or penicillin, each with a molecular weight of less than about 1,000 Daltons). However, analytes to be detected may be larger, including for instance immunoglobulin analytes (such as IgG, which is about 8 nm in length and about 160,000 Daltons). Analytes can be polyvalent or monovalent. Examples of analytes are disclosed, for example, in U.S. Pat. No. 4,299,916; U.S. Pat. No. 4,275,149; U.S. Pat. No. 4,806,311; U.S. Pat. No. 6,001,558; and PCT Publication No. 98/39657.

A “sample suspected of containing an analyte” is any sample of interest that could contain an analyte that can be used in the methods disclosed herein. The samples can be any biological fluid, such as but not limited to, serum, blood, plasma, cerebral spinal fluid, sputum, urine, nasal secretions, sweat, saliva, pharyngeal exudates, bronchoalveolar lavage fluids, or vaginal secretions. Fluid homogenates can also be utilized as samples, such as cellular homogenates or fecal suspensions. Samples can also be non-biological fluids such as environmental samples, plant extracts, soil extracts water samples. Typically a sample is in an aqueous form, or is an aqueous extract of a solid sample.

Analyte analog: a modified analyte that has structural similarity to the unmodified analyte and can bind to at least one analyte binding partner. An analyte analog includes, for example, a fragment of the full-length analyte or a mutated form of the analyte that is still recognized and bound by a specific binding partner. In certain embodiments of the invention, the analyte analog is an analyte-tracer conjugate, for instance a detectable analyte-tracer conjugate. Generally, the analyte analog can compete for biding of a specific binding partner with the unmodified analyte.

Antibody: a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

Antibodies can exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H} 1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially an Fab with part of the hinge region (see, Fundamental Immunology, W. E. Paul, ed., Raven Press, N.Y., 1993). While various antibody fragments are defined in terms of the digestion of an intact antibody, it will be appreciated that Fab′ fragments may be synthesized de novo either chemically or by utilizing recombinant DNA methodology. Thus, the term antibody as used herein also includes antibody fragments either produced by the modification of whole antibodies or synthesized de novo using recombinant DNA methodologies. Embodiments of the assay that use antibodies can use any form of the antibodies, such as the intact immunoglobulin or fragments thereof that retain desired specific binding characteristics.

Antibodies for use in the methods and devices of the invention can be monoclonal or polyclonal, but often will be monoclonal. Merely by way of example, such monoclonal antibodies can be prepared from murine hybridomas according to the classical method of Kohler and Milstein (Nature 256:495-497, 1975) or derivative methods thereof. Briefly, a mouse is repetitively inoculated with a few micrograms of the selected analyte compound (or a fragment thereof) over a period of a few weeks. In some instances, it will be beneficial to use an adjuvant or a carrier molecule to increase the immunogenicity and/or stability of the analyte in the animal system. The mouse is then sacrificed, and the antibody-producing cells of the spleen isolated. The spleen cells are fused by means of polyethylene glycol with mouse myeloma cells, and the excess un-fused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Meth. Enzymol. 70:419-439, 1980), and derivative methods thereof. Selected positive clones can be expanded and their monoclonal antibody product harvested for use. Detailed procedures for monoclonal antibody production are described in Harlow and Lane (Antibodies, A Laboratory Manual, CSHL, New York, 1988).

Monoclonal antibodies to different analytes are commercially available. For instance, a monoclonal antibody to estriol-3 is produced by Fitzgerald Industries International (Concord, Mass.; Cat. # 10-E37, Clone # M612039); likewise, Omega Biological, Inc. (Bozeman, Mont.) produces a monoclonal antibody to methamphetamine (Cat. # 100-11-183, Clone Met 2).

Antigenic: a chemical or biochemical structure, determinant, antigen or portion thereof that is capable of inducing the formation of an antibody.

Avidin/Streptavidin: The extraordinary affinity of avidin for biotin allows biotin-containing molecules in a complex mixture to be discretely bound with avidin. Avidin is a glycoprotein found in the egg white and tissues of birds, reptiles and amphibia. It contains four identical subunits having a combined mass of 67,000-68,000 daltons. Each subunit consists of 128 amino acids and binds one molecule of biotin. Extensive chemical modification has little effect on the activity of avidin, making it especially useful for protein purification.

Another biotin-binding protein is streptavidin, which is isolated from Streptomyces avidinii and has a mass of 60,000 daltons. In contrast to avidin, streptavidin has no carbohydrate and has a mildly acidic pI of 5.5. Another version of avidin is NeutrAvidin Biotin Binding Protein (available from Pierce Biotechnology) with a mass of approximately 60,000 daltons.

The avidin-biotin complex is the strongest known non-covalent interaction (Ka=10¹⁵ M⁻¹) between a protein and ligand. The bond formation between biotin and avidin is very rapid, and once formed, is unaffected by extremes of pH, temperature, organic solvents and other denaturing agents.

Although examples disclosed herein use streptavidin (SA) as a specific binding agent, the streptavidin could be substituted with other types of avidin. The term “avidin” is meant to refer to avidin, streptavidin and other forms of avidin that have similar biotin binding characteristics.

Bibulous: absorbent. Lateral flow test strips disclosed herein may be made of a bibulous matrix, such as a porous matrix, in which liquid flows by capillary action though the matrix. The support matrix of the device may be capable of either bibulous or non-bibulous lateral flow. Non-bibulous lateral flow refers to liquid flow in which all of the dissolved or dispersed components of the liquid are carried at substantially equal rates and with relatively unimpaired flow, laterally through the membrane or matrix, as opposed to bibulous flow in which different components flow at different rates. In certain examples disclosed herein, different components of liquid flow separate into distinct wave fronts that reach capture zones sequentially instead of simultaneously. The degree of separation of wave fronts can be controlled using a variety of factors, such as the pore size of the bibulous matrix (larger components move more slowly through the pores), weight (heavier components flow more slowly), and interactions with the substrate (hydrophobic, charge or other interactions between a component and the matrix alter migration rate). Bibulous flow is preferred in the embodiments disclosed herein to allow separation of the wave fronts as described in this specification.

Bibulous materials, such as untreated paper, cellulose blends, nitrocellulose, polyester, an acrylonitrile copolymer, rayon, glass fiber, and the like may also be employed as support matrix materials to provide non-bibulous flow. Especially preferred are microporous materials made from nitrocellulose, by which term is meant any nitric acid ester of cellulose. Thus suitable materials may include nitrocellulose in combination with carboxylic acid esters of cellulose. The pore size of nitrocellulose membranes may vary widely, but is preferably within 1 to 20 microns, preferably 8 to 15 microns. Bibulous flow can be enhanced by various methods that alter the binding properties of the support matrix, or by selectively placing different reagents in different support matrix environments, or position on the strip that restrict or enhance flow. To provide non-bibulous flow, these materials may be treated with blocking agents that may block the forces which account for the bibulous nature of bibulous materials. Suitable blocking agents include bovine serum albumin (BSA), methylated bovine serum albumin, whole animal serum, casein, and non-fat dry milk. Certain localized regions of a test strip may be blocked without completely abolishing differential flow on the test strip.

Binding affinity: a term that refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the invention that these constants be measured or determined. Rather, affinities as used herein to describe interactions between molecules of the described methods and devices are generally apparent affinities (unless otherwise specified) observed in empirical studies, which can be used to compare the relative strength with which one molecule (such as an antibody or other specific binding partner) will bind two other molecules (such as an analyte and an analyte-tracer conjugate). The concepts of binding affinity, association constant, and dissociation constant are well known.

Binding domain: the molecular structure associated with that portion of a receptor that binds ligand. More particularly, the binding domain may refer to a polypeptide, natural or synthetic, or nucleic acid encoding such a polypeptide, the amino acid sequence of which represents a specific (binding domain) region of a protein, which either alone or in combination with other domains, exhibits specific binding characteristics that are the same or similar to those of a desired ligand/receptor binding pair. Neither the specific sequences nor the specific boundaries of such domains are critical, so long as binding activity is exhibited. Likewise, used in this context, binding characteristics necessarily includes a range of affinities, avidities and specificities, and combinations thereof, so long as binding activity is exhibited.

Binding partner: any molecule or composition capable of recognizing and specifically binding to a defined structural aspect of another molecule or composition. Examples of such binding partners and corresponding molecule or composition include antigen/antibody, hapten/antibody, lectin/carbohydrate, apoprotein/cofactor and biotin/avidin (such as biotin/streptavidin).

Biotin binding protein: A protein (such as a specific binding protein) that binds biotin with sufficiently great affinity for an intended purpose. Examples of biotin binding proteins are well known in the art, and include avidin, streptavidin, NeutrAvidin, and monoclonal antibodies or receptor molecules that specifically bind biotin. In the context of this disclosure, streptavidin could be replaced with all biotin-binding proteins.

Chelator (chelating resin): a composition that binds divalent cations. The binding can be reversible or irreversible. Binding of divalent cations generally renders them substantially unable to participate in chemical reactions with other moieties with which they come in contact. Chelators are well known and include ethylenediamine tetraacetate (EDTA), sodium citrate, ethyleneglycol-bis(β-oxyethylenenitrilo)-tetraacetic acid (EGTA), trans-1,2-diaminocyclohexane-N,N,N′,N′-tetraacetic acid (CDTA), nitriloacetic acid (NTA), resins that contain moieties that bind divalent cations and the like. Chelators that remain in solid phase in the solution in question are referred to as chelating resins. Chelating resins can be used to pull the subject ion (e.g., Ca²⁺) out of solution. Chelating resins include, but are not limited to, chelex resins containing iminodiacetate ions, resins containing free base polyamines, aminophosphonic acid, and the like.

Conjugate: In the context of this disclosure, the conjugate refers to different moieties bound to one another, for example by covalent bonds. An example of a conjugate is an analyte or antibody tagged with a specific binding agent, such as biotin. Biotinylation of a substrate (such as an antibody or analyte) is routinely achieved in the art by reacting a free carboxyl group on biotin with an amine group on a protein, such as an amine group found in an antibody or protein analyte/analog. Although biotinylation of a substrate is sometimes referred to as “labeling” in the prior art, the label in the context of this application instead refers to providing a visually detectable agent.

Detect or determine an analyte: An analyte is “detected” when its presence is ascertained or discovered. “Determination” of an analyte refers to detecting an amount/concentration (either approximate or exact) of the analyte. Hence “detection” is a generic term that includes either ascertaining its presence or determining an amount/concentration (since determining an amount also indicates the presence of the analyte).

Detectable label: A label capable of providing a signal that can indicate to an observer the presence of the label. Examples of detectable labels include colored particles (such as latex spheres) and fluorescent molecules.

Flow path: Typically, the support matrix will define a flow path from a sample application zone through at least two capture zones, and optionally to an absorbent zone. The flow path is generally axial, although other configurations are acceptable and may be preferred for some embodiments. The flow path may be superficially on the surface of a substrate (for example on a non-bibulous substrate that substantially excludes liquid flow through the matrix of the substrate), or substantially entirely within and through the substrate itself (for example, through the porous structure of a substrate that does not exclude liquid from it). For example radial, multi-lane, undulating or circular flow paths are useful in test devices that can simultaneously detect the presence of multiple analytes in a sample. Within the overall flow path toward the capture zones, there may be separate selective paths that individual chemical components may take to achieve differential migration of the individual components for the purpose of separation of components, or temporal delay of reaction. Thus there may be several wavefronts within the overall flow path.

Freely suspendable: a state of permeation or reversible surface adherence. Substances that are freely suspendable are diffusively bound on a surface such that they are not immobilized within or upon a support matrix but are capable of being mixed or suspended in liquids placed on the support matrix. Such suspended substances are capable of migrating with liquids moving along the support matrix. Generally lateral flow devices utilize components that are freely suspendable, see for example PCT Publication No. WO 98/39657.

Immobilized: Certain binding partners disclosed herein are immobilized to a flow matrix such as a test strip. Immobilization in the drawings is indicated by a line touching the substrate, in contrast to mobile binding partners which are illustrated without a line connecting them to the test strip. Immobilized binding partners are associated with the flow matrix in a manner that substantially localizes the binding partner to the location in which it is placed. Immobilization can be achieved using any of a variety of techniques, for example by activating the matrix prior to placing the binding partner on it. The particular methods depend on the nature of the bibulous matrix and the particular binding pair member being immobilized. For example, a specific binding partner can be immobilized through activation of a substrate by carbonyldiimidazole, glutaraldehyde, succinic acid, or cyanogen bromide. Alternatively, particles having an immobilized specific binding pair member may be used to immobilize the specific binding pair member on the capture zone. Exemplary of such particles are latex beads made of polystyrene, polyacrylates and polyacrylamides that are of a sufficient size and/or weight to not migrate within the test strip. The particles are capable of non-diffusive attachment of the specific binding pair member by covalent or non-covalent binding, for example through functional groups such as carboxylic acids, aldehydes, amines, thiols, hydroxyls and the like.

Immunogen: a chemical or biochemical structure, determinant, antigen or portion thereof, that elicits an immune response, including, for example, polylysine, bovine serum albumin and keyhole limpet hemocyanin (KLH).

Label: a marker attached to a molecule to identify or otherwise detect it. A detectable label may be, for example, any molecule or composition that is detectable by spectroscopic, photochemical, biochemical, immunochemical, electrical, visual (including optical) or chemical means. Examples of labels include enzymes, colloidal gold particles, carbon particles, colored latex particles, fluorescent molecules, and others such as those disclosed in U.S. Pat. Nos. 4,275,149; 4,313,734; 4,373,932; and 4,954,452. The disclosure of those particles is incorporated by reference herein to provide additional examples of labels.

The attachment of a label to a target being labeled can be through covalent bonds, adsorption processes, hydrophobic and/or electrostatic bonds, as in chelates and the like, or combinations of these bonds and interactions and/or may involve a linking group. In particular examples disclosed herein, the label is a colored agent (such as colored latex, gold or carbon particle or a fluorescent molecule) to which a binder (such as streptavidin) is attached. The label can migrate independently of the target (such as the conjugate) that it is intended to label, but the label associates with the target during the assay.

Some forms of labeling include the use of radioactive isotopes, dyes, fluorescent labels, and enzyme labels. When detecting an enzyme label, a substrate may be supplied to activate a color change that provides the detected signal.

A “direct label” is one that is always detectable by itself (such as a colored particle or dye); an “indirect label” is one that does not provide a detected signal by itself. An indirect label may need to be activated (for example by addition of an enzyme substrate) to provide a signal, or submitted to a detector (such as illumination with ultraviolet light or exposure to a radiation detector).

Lateral flow device: devices that include bibulous or non-bibulous matrices capable of transporting analytes and reagents to a pre-selected site. Many such devices are known, in which the strips are made of nitrocellulose, paper, cellulose, and other bibulous materials. Non-bibulous materials can be used, and rendered bibulous by applying a surfactant to the material. The bibulous matrices typical are porous strips through which liquid is transported. The porous structure of such strips provides a flow path through the matrix for conducting the flow of liquid.

Lateral flow chromatography strip: a test strip used in lateral flow chromatography, in which a test sample fluid, suspected of containing an analyte, flows (for example by capillary action) through the strip (which is frequently made of materials such as paper or nitrocellulose). The test fluid and any suspended analyte can flow along the strip to a detection zone where the presence or absence of the analyte is signaled.

Linking group: a chemical bridge between two compounds, for instance a compound and a label (such as a colored particle and a conjugate). In particular examples disclosed herein, the linking group includes a binding pair, such as biotin/avidin (such as biotin/streptavidin), carbohydrate/lectin, or a ligand/receptor, in which one of the binding pair members is present on the label and the other member is present on the conjugate.

Porosity: percentage of a substrate that is air. For example, a membrane with a porosity of 0.7 is 70% air. The porosity of the lateral flow substrates disclosed herein can be altered to change flow rate of liquid through the substrate.

Positive/direct reporting: an increase in the reporting or detection signal with increasing analyte concentration.

Sample-receiving zone: An area of a test strip on which sample may be placed, for example to perform a lateral flow assay. In some disclosed embodiments the sample-receiving zone is spaced from, and upstream from the mobilization zone. However in other embodiments it may have a common border with the mobilization zone. The sample-receiving zone is illustrated in the drawings as spaced from the mobilization zone; that convention is only for purposes of simplified illustration in the drawings.

Specific binding partner: a member of a pair of molecules that interact by means of specific, non-covalent interactions that depend on the three-dimensional structures of the molecules involved. Exemplary pairs of specific binding partners include antigen/antibody, hapten/antibody, ligand/receptor, nucleic acid strand/complementary nucleic acid strand, substrate/enzyme, inhibitor/enzyme, carbohydrate/lectin, biotin/avidin (such as biotin/streptavidin), and virus/cellular receptor. The methods and devices disclosed herein can be used for any analyte for which a specific binding partner exists.

The phrase “specifically binds to an analyte” (or “specifically immunoreactive with” when referring to an antibody) refers to a binding reaction which is determinative of the presence of the analyte in the presence of a heterogeneous population of molecules such as proteins and other biologic molecules. A cellular receptor is, for example, capable of specifically binding to an analyte. In immunoassay conditions, the specified antibodies bind to a particular analyte and do not bind in a significant amount to other analytes present in the sample. A variety of immunoassay formats may be used to select antibodies specifically immunoreactive with a particular analyte. For example, solid-phase ELISA immunoassays are routinely used to select monoclonal antibodies specifically immunoreactive with a protein. See Harlow and Lane, Antibodies, A Laboratory Manual, CSHP, New York (1988), for a description of immunoassay formats and conditions that can be used to determine specific immunoreactivity.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” It is further to be understood that all molecular weight or molecular mass values are approximate, and are provided for description. Although methods and materials similar or equivalent to those described herein can be used in the practice or testing of this disclosure, suitable methods and materials are described below. In addition, the materials, methods, and examples are illustrative only and not intended to be limiting.

The disclosure is illustrated by the following non-limiting Examples.

A lateral flow test strip is described herein that provides a device for conducting a lateral flow immunoassay, and enables a method of using the test strip to perform the test. As discussed in greater detail below, a feature of the disclosed assay is a label that migrates independently of a conjugate that binds at a primary or secondary capture zone, to label the conjugate after it has bound at the capture zone.

### Example 1

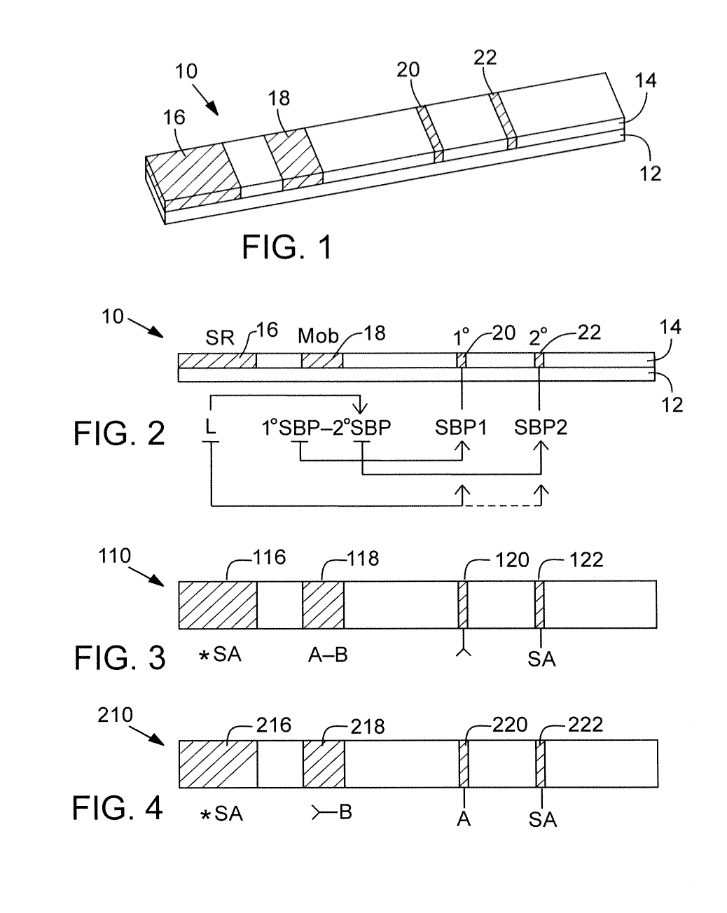

FIG. 1 illustrates a particular embodiment of a narrow, porous, elongated, lateral flow test strip 10 that includes a rigid or semi-rigid plastic support backing 12 on which is mounted a bibulous layer 14. Bibulous layer 14 is made, for example, of nitrocellulose and defines a porous flow path through test strip 10 from a sample receiving zone 16 through a mobilization zone 18 to a primary capture zone 20 and a secondary capture zone 22. Although zones 16, 18, 20 and 22 are illustrated as discrete rectangular areas that are separated from one another, this depiction is only for purposes of illustration. The zones can be contiguous, spaced by any distance, or of any convenient shape or size. Additional zones may also be included on the test strip, such as a filtration zone between sample receiving zone 16 and mobilization zone 18 to remove impurities from a liquid sample applied to the test strip. In addition a superabsorbent zone may be placed downstream of secondary capture zone 22 (at the distal end of the test strip) to absorb the distal flow of liquid after it traverses the test strip. These and other additional zones are not shown in the drawings for purposes of clarity of the drawings.

For example, label L can be placed at different positions on test strip 10 where it can reach the primary capture zone after the conjugate. For example, label L may be anywhere upstream of the conjugate, such as between sample receiving zone 16 and mobilization zone 18, or adhered to layer 14 with a localized delayed release process, such as an agent that slows its migration relative to a migration rate of the conjugate in the liquid sample. A variety of techniques may be used to control migration of label L so that it migrates behind the conjugate, and these techniques may be combined in different combinations. Hence the relative weight of label L compared to the conjugate can slow its relative migration rate; a heavy metal particle (such as gold sol) can be used to increase the label weight and slow its migration rate. Alternatively, the label can be a very large molecule (relative to pore size of the substrate) that slows the migration rate of the label relative to the conjugate. For example, the label can include a colored latex particle that is bound to the specific binding agent (such as an avidin, for example streptavidin) by a long linker, such as bovine serum albumin (BSA). Alternatively, the specific binding agent (such as streptavidin) can be attached directly to the colored latex particle, but one or more other long or heavy tails can be attached to the colored particle to increase its weight or effective size relative to the pores through which it migrates. In addition the movement of the label or conjugate may be selectively slowed by placement onto highly bibulous materials below or above the main support matrix.

In other embodiments the label is placed on the substrate in a position that retards its migration rate relative to the conjugate. For example, the label can be placed entirely beneath the surface of the substrate where it encounters more resistance to flow than it would on the surface, where the conjugate is present. Placement below the surface can be achieved in one example by applying the label to the back surface of the substrate instead of the top surface; the conjugate is then applied to the top surface to assure its migration with less resistance along the surface of the test strip. In other examples, the label is placed on the surface of the test strip but below (for example completely covered by) an overlying sample receiving pad, so that liquid flows from the pad into and along the more superficial layers of the substrate. The label, however, must be solubilized and mobilized before it is released from the substrate for delayed migration along the substrate. In some embodiments, the pad completely covers the label in the substrate, and the pad extends over the label by a distance of at least 2 mm, for example 2-10 mm, to further retard a rate of migration of the label through the substrate. In some embodiments, the pad does not cover the conjugate, but in other embodiments the pad also covers the conjugate partially or completely to delay migration of the conjugate. In some embodiments, the pad overlaps the label more than the conjugate, so that migration of the label is slowed more than migration of the conjugate.

Delayed release of the label L can also be achieved by placing the label in a localized area or zone (such as a sub-section of the sample receiving zone) where a delayed release agent is present. Since the delayed release agent is selectively present around label L, it selectively slows the “release” of label L from the substrate relative to the faster “release” of the conjugate that is not retarded by a delayed release agent. Examples of such delayed release agents that can be selectively concentrated around label L, for example in sample receiving zone 16, include sucrose (about 5-50%, mannitol (about 5-30%), glycerol (about 1-15%), polyvinyl alcohol (PVA)(about 0.1-5%), polyvinyl pyrrolidone (PVP)(about 0.1-5%) and mixtures thereof. Delayed release is achieved with these treatments by increasing the local viscosity surrounding the label. This viscous zone moves more slowly than the liquid that moves ahead of and around the viscous zone. An example of this which is used in lateral flow applications is described in U.S. Pat. No. 6,306,642 which uses cyclodextrin as a delayed release agent to slow migration of an enzyme conjugate.

Another approach that can be used to control the relative rates of the label and conjugate is to select the polarity or charge of the label so that its rate of migration through the substrate is retarded. For example, the charge found on most colloidal particles is negative, as a result of the chelation of anionic organic molecules (usually citrate) used in the preparation of gold sol. As a result, chemically modifying the sample pad matrix to convert it from a slightly negative silica material, to a strongly positive material, will significantly retard the movement of the chelated gold, due to an “ion exchange, charge-charge effect.” When the glass fiber matrix is reacted with 3-aminopropyl-trimethoxysilane at acid pH, the silica hydroxyls are converted to silyl esters that contain positively charged amino groups. When a negatively charged colloidal gold conjugate is impregnated onto and then subjected to flows through this matrix, its movement is retarded by charge-charge interactions.

The migration rate of label L relative to the conjugate can also be slowed by placing label L and the conjugate on the substrate in a position (for example separated by a sufficient distance or depth) that the conjugate migrates in a first wave front to the primary capture zone ahead of a second wave front in which label L migrates to the primary capture zone. The distance of separation on the substrate may be determined, for example, by the physical and functional characteristics of the label L, conjugate, substrate, and other factors. For example the distance of separation on the test strip between the label and conjugate can be varied depending on characteristics of the label and matrix, such as weight, size, polarity or charge of the label, the presence of a delayed release agent on the matrix, the pore size of the matrix, etc.

In one example, the label and conjugate are placed on the test strip, separated by a distance that maintains a separation of the wave fronts in which the label and conjugate flow. The separation of the wave fronts is maintained until the label wave front reaches a pre-selected position, such as the primary capture zone. In preferred embodiments, the conjugate wave front reaches the primary capture zone before the label wave front, so that the conjugate has an opportunity to interact with the primary capture zone before the label interacts with the conjugate or the primary capture zone. In other embodiments, the separation of the conjugate and label wave fronts is maintained until the conjugate reaches the secondary capture zone. The distance on the test strip between the label and conjugate can be less if the characteristics of the matrix or label substantially retard migration of the label relative to migration of the conjugate. However if the matrix or label provide less retardation of flow rate of the label, then the distance of separation between the label and conjugate is greater to assure more complete separation of the wave fronts. Further details about the separation of wave fronts is provided in Example 10.

### Example 2

As schematically illustrated in FIG. 2, a mobilizable detectable label L is present in sample receiving zone 16, a mobilizable conjugate 1° SBP-2° SBP is present in mobilization zone 18, an immobilized first specific binding partner SBP1 is present in primary capture zone 20, and an immobilized second specific binding partner SBP2 is present in secondary capture zone 22. Immobilized reagents are illustrated with a line connecting them to test strip 10 in FIG. 2. As schematically illustrated by the connecting arrows, the primary specific binding partner 1° SBP of the conjugate specifically binds the first specific binding partner SBP1 in primary capture zone 20, and the secondary specific binding partner 2° SBP specifically binds the second specific binding partner SBP2 in secondary capture zone 22. The label L is capable of binding the secondary specific binding partner 2° SBP of the conjugate.

In use, a liquid sample that may contain the analyte of interest is applied to sample receiving zone 16. The liquid sample is absorbed by the bibulous matrix of layer 14 and migrates along the distal path of flow through the mobilization zone 18 and primary and secondary capture zones 20, 22. The liquid mobilizes label L which begins to flow behind the leading edge of the advancing liquid front, which reaches mobilization zone 18 to mobilize conjugate 1° SBP-2° SBP and move it toward primary capture zone 20. In the absence of analyte in the sample the 1° SBP of the conjugate freely binds to SBP1 in primary capture zone 20. However if analyte is present in the sample it inhibits the binding of 1° SBP to SBP1 such that the conjugate passes through primary capture zone 20 to secondary capture zone 22, where the 2° SBP component of the conjugate binds to SBP2.

It is advantageous for the assay to be designed such that the conjugate migrates along the test strip behind the analyte, at least until the analyte reaches the primary capture zone. This differential migration permits the analyte (if present) to substantially occupy the binding sites of the primary capture zone before the conjugate can bind to them, as disclosed in greater detail in U.S. Pat. No. 6,699,722, the disclosure of which is incorporated by reference herein. Differential migration of the analyte and conjugate can also be achieved using the same methods described herein for achieving differential migration of the label and conjugate, including placing the conjugate a sufficient distance from the primary capture zone that the wave front containing the conjugate does not overtake the analyte before the analyte reaches the primary capture zone.

### Example 3

A particular competitive assay example of the delayed labeling method is schematically illustrated in FIG. 3, in which a path of liquid flow is provided along a test strip 110 from a sample receiving zone 116, through a mobilization zone 118 to a primary capture zone 120 and a secondary capture zone 122. Label *SA is a mobilizable colored particle (such as gold sol) coated with a biotin binding protein such as an avidin such as streptavidin (SA), and located in sample receiving zone 116. A mobilizable conjugate A-B (analyte or analyte analog bound to biotin) is present in mobilization zone 118. The 1° SBP of the conjugate is analyte or an analog of the analyte A and the 2° SBP is biotin B bound to the 1° SBP. Primary capture zone 120 contains immobilized antibody that specifically binds to the analyte or analog, and secondary capture zone 122 contains immobilized streptavidin. Liquid sample applied to sample receiving zone 116 mobilizes the streptavidin/gold label *SA which is sufficiently heavy and/or large that it flows though the pores of the matrix behind the leading front of the liquid as the liquid migrates in the direction of flow distally down test strip 110. Label *SA has been spaced on the test strip a sufficient distance from conjugate A-B that it does not overtake conjugate A-B until after the conjugate has reached primary capture zone 120. The wave front of the liquid reaches conjugate A-B before label *SA reaches conjugate A-B, and the liquid mobilizes conjugate A-B which continues to move ahead of label L to primary capture zone 120.

If analyte is present in the sample, the analyte A substantially occupies the binding sites of the antibodies immobilized in primary capture zone 120, such that binding of the A portion of conjugate A-B in the primary capture zone is inhibited or prevented. This promotes migration of conjugate A-B through the primary capture zone to the secondary capture zone where the biotin B portion of conjugate A-B binds to the immobilized streptavidin SA. Label *SA then reaches the bound conjugate A-B and streptavidin portion SA of label *SA also binds to biotin B portion of the conjugate to provide a visible signal from the secondary capture zone that indicates the presence of analyte in the sample.

If analyte is absent from the sample, or present below a level of detection, portion A of conjugate A-B occupies binding sites of the antibodies immobilized in primary capture zone 120. When the streptavidin/gold label subsequently reaches the primary capture zone, the streptavidin binds to the biotin B portion of conjugate A-B to localize the gold label in the primary capture zone and provide a visible signal that conjugate A-B is bound there.

The separation of the conjugate and the label avoids the problems inherent in attaching the analyte or analog directly to the colored particle. For example, when the analyte or analog is linked directly to the colored particle, it is more difficult to achieve high sensitivity of the assay when testing for low concentration analytes such as FSH. The solid phase reactivity of the smaller conjugate is greater than the same conjugate if it were attached to a larger particle. It can be metered and controlled better than the particle based conjugate. Secondly, the label L can be added in excess to drive the reaction without concern about metering the label, which is often more difficult to precisely dispense and control.

### Example 4

An example of another assay that incorporates the delayed labeling technique is shown in the test strip 210 of FIG. 4 in which a path of liquid flow proceeds distally from sample receiving zone 216 through mobilization zone 218 to primary capture zone 220 and secondary capture zone 222. Label *SA is a mobilizable colored particle (such as gold sol) coated with streptavidin (SA) that is placed in sample receiving zone 216. The mobilizable conjugate is present in mobilization zone 218, and 1° SBP of the conjugate is antibody that specifically binds the analyte A while the 2° SBP is biotin B bound to the 1° SBP. Primary capture zone 220 of test strip 210 contains immobilized analyte or analyte analog A that specifically binds with the antibody of the conjugate, and secondary capture zone 222 contains immobilized streptavidin SA. Liquid sample applied to sample receiving zone 216 mobilizes the streptavidin/gold label *SA which is sufficiently heavy that it flows behind the leading front of the liquid as the liquid moves in the direction of flow distally down test strip 210. The wave front of the liquid reaches the conjugate ahead of label *SA, and mobilizes conjugate which moves ahead of label *SA to primary capture zone 220.

If analyte is present in the sample, the analyte A occupies the binding sites of the antibody portion of the conjugate, such that binding of the conjugate to analyte/analog in primary capture zone 220 is inhibited or prevented. Conjugate therefore continues to migrate ahead of label *SA through primary capture zone 220 to secondary capture zone 222 where the biotin B portion of conjugate binds to the immobilized streptavidin SA. Label *SA then reaches the bound conjugate in secondary capture zone 222, and the streptavidin portion of label *SA also binds to biotin B portion of the conjugate to provide a visible signal from secondary capture zone 222 that indicates the presence of analyte in the sample.

If analyte is absent from the sample, or present below a level of detection, the antibody portion of the conjugate binds to immobilized analyte/analog A in primary capture zone 220. When the streptavidin/gold label *SA subsequently reaches primary capture zone 220, the streptavidin binds to the biotin B portion of the conjugate to localize the gold label in primary capture zone 220 and provide a visible signal that conjugate A-B is bound there.

The separation of the conjugate and the label avoids the problems inherent in attaching the antibody directly to the colored particle. For example, when the antibody is coated on or linked directly to the colored particle, multiple antibodies (providing for example 50-70 or more active antibody sites on a 40 nm particle) are present on each gold particle. These multiple binding sites on the colored particle allow multiple analyte molecules in the sample to bind to the conjugate, which reduces the efficiency by which analyte molecule blocks the conjugate from attaching to the primary capture zone. For example, if there are 70 antibody binding sites on the conjugate, it may require 35 analyte molecules to convert 50% of each conjugate molecule to a “bound” state that inhibits it binding in the primary capture zone.

However, separating the label from the conjugate provides much greater control over the stoichiometry of the reactions. Since the conjugate can contain a single antibody, there can be a low ratio of about 1:1 or 1:2 between the conjugate and analyte in the sample (as compared to a ratio of (35-70): 1 or more when an antibody coated particle is used). In this controlled stoichiometric reaction, far fewer analyte molecules interact with the conjugate. It is also possible to better control the ratio of primary to secondary specific binding partner (antibody to biotin in this example) in a specified ratio, such as less than 3:1, for example 2:1 or 1:1. If the conjugate migrates with the sample front, it may not have time to react, or the volume of analyte solution to react with, in order to obtain maximal sensitivity. Therefore it is advantageous in some embodiments to delay migration of the conjugate to control sensitivity. Migration of the conjugate can be delayed using any of the techniques described herein with respect to migration of the label. Migration may be delayed to a greater extent with the label than the conjugate in certain embodiments so that the conjugate reaches the primary capture zone before the label. As noted in Example 3 above, the solid phase reactivity of the smaller conjugate is greater than the same conjugate were it attached to a larger particle. The conjugate can be metered and controlled better than the particle based conjugate, and the label can be added in excess to drive the reaction without needing to meter label.

### Example 5

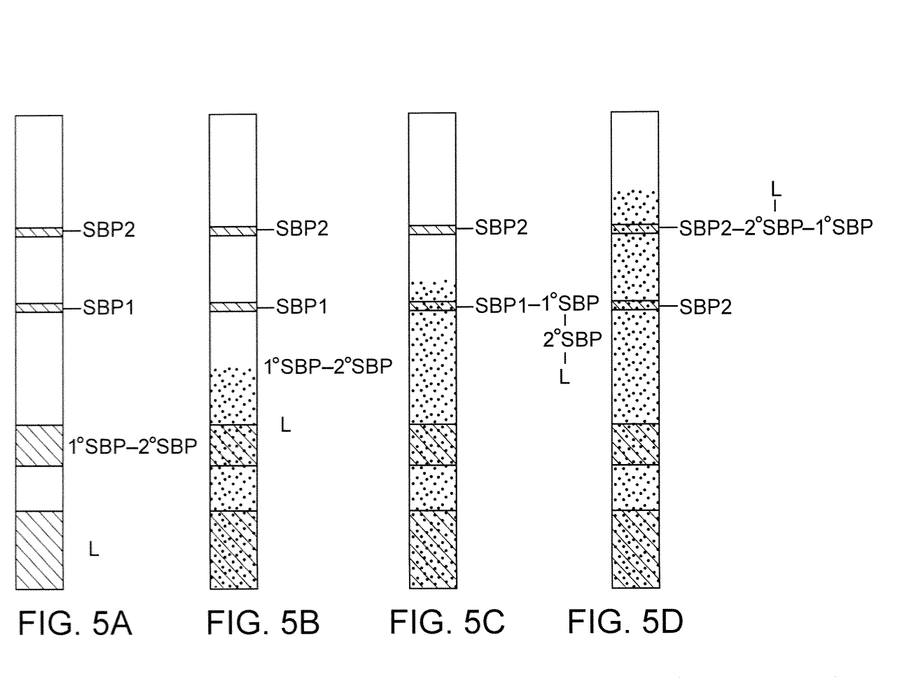

FIG. 5 is another schematic drawing that illustrates the test strip as the wave front of the liquid sample migrates distally through the strip. FIG. 5A shows the mobile label L in the sample receiving zone, the mobile conjugate 1° SBP-2° SBP in the mobilization zone, the immobilized SBP1 in the primary capture zone and the immobilized SBP2 in the secondary capture zone. FIG. 5B shows the relative position of the components after liquid sample has been applied to the sample receiving zone and the liquid has flowed through the mobilization zone but has not yet reached the primary capture zone. Both label L and conjugate 1° SBP-2° SBP have moved from their original locations, but conjugate 1° SBP-2° SBP is migrating in advance of label L. In FIG. 5C, conjugate 1° SBP-2° SBP has bound to SBP1 through 1° SBP, and label L has then subsequently bound to 2° SBP of the conjugate to provide a visible signal from the primary capture zone. If conjugate 1° SBP-2° SBP is unable to bind to the primary capture zone, or is inhibited from doing so, FIG. 5D shows that the conjugate continues to migrate ahead of label L to the secondary capture zone where 2° SBP binds to SBP2 label L then subsequently binds to 2° SBP to provide a visible signal from the secondary capture zone.

### Example 6

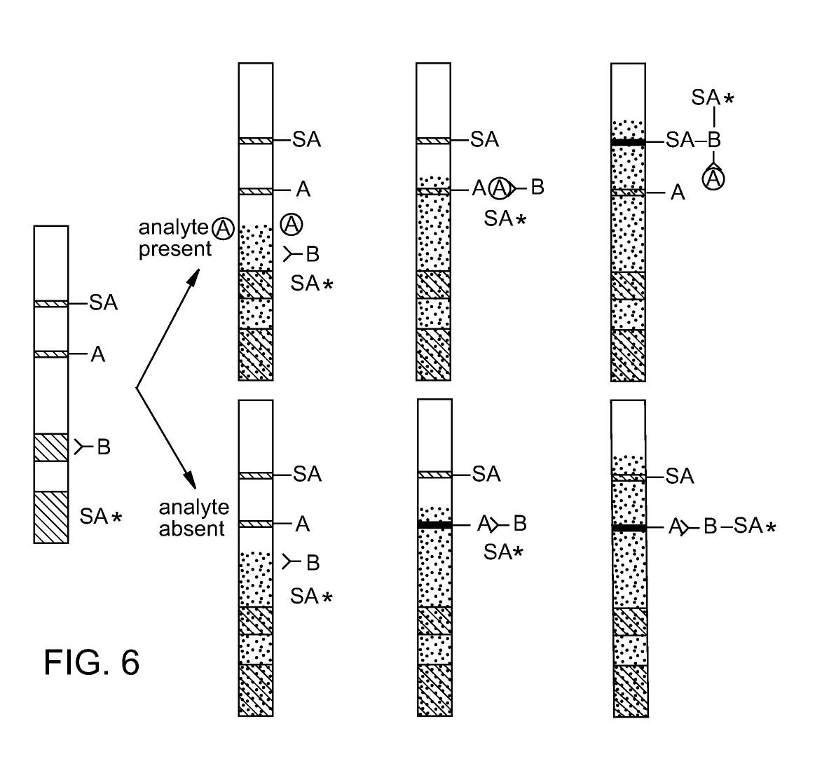

FIG. 6 schematically illustrates the competitive assay in which analyte is present (top row) or absent (bottom row). The illustrated test strip has a sample receiving zone in which mobilizable label *SA is located, a mobilization zone in which mobilizable conjugate is present, a primary capture zone in which analyte or analyte analog is immobilized, and a secondary capture zone in which streptavidin SA is immobilized. The movement of the label, conjugate and analyte are indicated schematically in FIG. 6, in which relative positions of these components are indicated in the drawing as the wave front of liquid sample moves distally along the path of flow.

If analyte \textcircled{A} is present in the sample (FIG. 6, top row), the analyte \textcircled{A} migrates in a wave front in advance of the label *SA and binds to the antibody of the conjugate before the conjugate reaches the primary capture zone so that movement of the conjugate is promoted to continue on to the secondary capture zone where the biotin component binds the immobilized streptavidin, and subsequently is in turn bound by the *SA label to provide a signal from the secondary capture zone. If the analyte \textcircled{A} is absent from the sample (FIG. 6, bottom row), the antibody of the conjugate binds to the immobilized analyte/analog A, which is subsequently bound by the *SA label to provide a signal from the primary capture zone.

Although FIG. 6 illustrates the primary and secondary capture zones as providing either an all-or-nothing signal, it is often the case that some residual color is left in the primary capture zone even when high levels of analyte are present. In some embodiments of the assay, differences in color between the primary and secondary capture zones can even be used to provide additional data. For example, in a specific embodiment of the assay of FIG. 6, the pattern of signals can be interpreted as follows:

| 1° Capture Zone | 2° Capture Zone | Interpretation |
|---|---|---|
| No signal | No signal | Defective assay or unused strip |
| Greater signal | No signal | Analyte absent |
| Lesser signal | Greater signal | Analyte present |
| No signal | Signal | Analyte present in large amount |

Other combinations of signals are not excluded by the examples listed above.

### Example 7

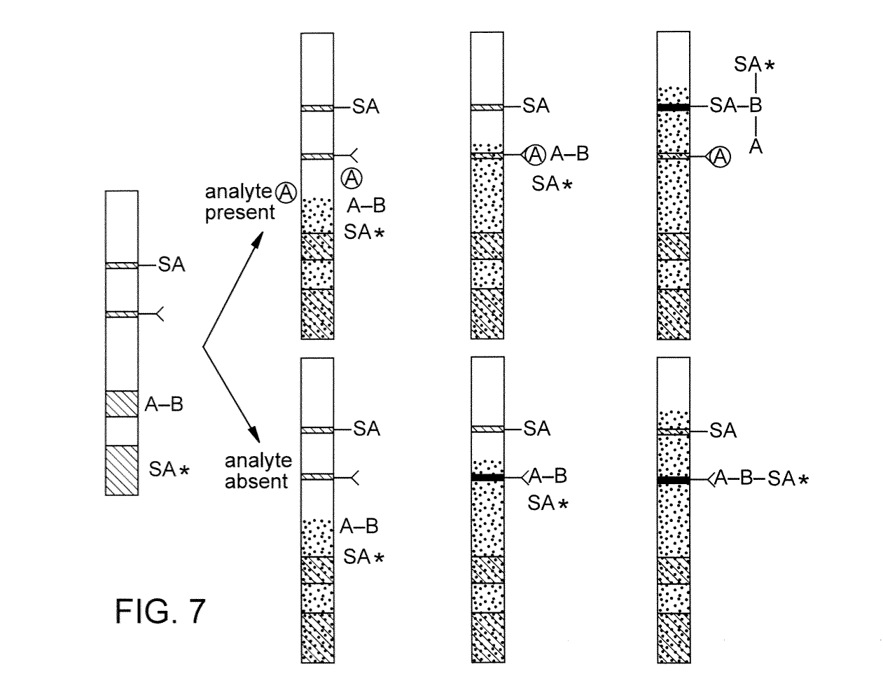

FIG. 7 schematically illustrates an assay in which analyte is present (top row) or absent (bottom row). The illustrated test strip has a sample receiving zone in which mobilizable label *SA is located, a mobilization zone in which mobilizable conjugate A-B is present, a primary capture zone in which antibody that specifically binds analyte or analyte analog is immobilized, and a secondary capture zone in which streptavidin SA is immobilized. The movement of the label, conjugate and analyte are indicated schematically in FIG. 7, in which relative positions of these components are indicated in the drawing as the wave front of liquid sample moves distally along the test strip.

If analyte \textcircled{A} is present in the sample above a detection level, analyte \textcircled{A} migrates in a wave front in advance of the label *SA and binds the immobilized antibody in the primary capture zone, so that conjugate binding to the primary capture zone is inhibited and the conjugate continues on to the secondary capture zone where its biotin component is bound by the immobilized streptavidin SA. The label *SA then subsequently reaches the secondary capture zone where it binds with the biotin component of the conjugate to provide a visible signal from the secondary capture zone. If analyte \textcircled{A} is absent from the sample or present only below a detection level, the analyte/analog component of the conjugate binds to the immobilized antibody in the primary capture zone. The label *SA subsequently reaches the primary capture zone where it binds to the biotin component of the conjugate to provide a visible signal from the primary capture zone.

As in Example 6, for purposes of illustration this Example 7 illustrates the primary and secondary capture zones as providing either an all-or-nothing signal. However it is often the case that some residual color is left in the primary capture zone even when high levels of analyte are present. In some embodiments of the assay, differences in color between the primary and secondary capture zones can even be used to provide additional data. For example, in a specific embodiment of FIG. 7, the pattern of signals can be interpreted as follows:

| 1° Capture Zone | 2° Capture Zone | Interpretation |
|---|---|---|
| No signal | No signal | Defective assay or unused strip |
| Greater signal | No signal | Analyte absent |
| Lesser signal | Greater signal | Analyte present |
| No signal | Signal | Analyte present in large amount |

Other combinations of signals are not excluded by the examples listed above.

### Example 8

### Specific Embodiment

One particular example of an assay of the type shown in FIGS. 4 and 6 is a competitive beta-human chorionic gonadotropin (β-hCG) lateral flow assay in which the test strip is made of nitrocellulose (Millipore HF 135). In this example the label is streptavidin conjugated to a colloidal gold particle having a diameter of about 40 nm. This label is a visually detectable heavy label that is positioned on the test strip upstream from but near the sample application zone. The conjugate is mouse anti-beta-hCG monoclonal antibody to which biotin has been covalently attached, in a ratio of about 3 biotins:antibody. The conjugate is placed on the test strip downstream from the label, for example in the mobilization zone.

The first specific binding partner in the primary capture zone is immobilized whole molecule hCG (5000 IU/mg), striped directly on to the nitrocellulose test strip in an amount of about 1000 ng/band. The second specific binding partner in the secondary capture zone is streptavidin (available from Jackson ImmunoResearch laboratories, Inc., West Grove, Pa., USA), which is striped directly on the nitrocellulose in an amount of approximately 250 ng/band.

If β-hCG is present in a liquid sample applied to the sample receiving zone, it binds to the antibody portion of the conjugate to form a complex. The hCG/conjugate complex reaches the primary capture zone prior to the label, and the already bound antibody of the complex inhibits binding of the complex to the hCG in the primary capture zone. The complex instead passes through the primary capture zone to the secondary capture zone, where the biotin of the conjugate binds the immobilized streptavidin. The label later reaches the secondary capture zone where the streptavidin of the label binds to the biotin of the conjugate to provide a detectable signal from the secondary capture zone that indicates the analyte has been detected.

The test can be made quantitative by developing a standard curve which measures the quantity of label in both primary and secondary capture zones, using either digital photography combined with software driven light reflectance readings (Adobe Photoshop), or a reflectometer that will quantitate lateral flow strips. The standard curve will consist of the ratio of secondary signal/Primary signal versus mIU/ml hCG added.

### Example 9

### Labels

In certain examples, the labels include a colored particle such as colloidal gold, a fluorescent compound, a latex particle, a carbon particle, a dye or an enzyme. However, a variety of labeling methods can be used in the present methods, including calorimetric, chemiluminescent, fluorescent and other known labeling techniques. The methods are preferably directly visible, and these include but are not limited to particulate labels such as dyed latex beads, erythrocytes, liposomes, dyes sols, metallic and nonmetallic colloids, stained microorganisms and other such labels known to those skilled in the art. Suitable labels such as colloidal metals, e.g. gold, and dye particles are disclosed in U.S. Pat. Nos. 4,313,734 and 4,373,932; the disclosure about these particles in both patents is incorporated by reference. Non-metallic colloids, such as colloidal selenium, tellurium and sulfur are disclosed in U.S. Pat. No. 4,954,452, incorporated by reference. Dyed microorganisms as labels are disclosed in U.S. Pat. No. 5,424,193, EP 0 074 520 and British Patent No. GB 1,194,256, all incorporated by reference. Dyed latex particles are disclosed in U.S. Pat. No. 4,703,017, incorporated by reference.

The intensity of an accumulated label in the capture zones can be correlated with analyte concentration in the sample by comparing the visible intensity of the signal to a reference standard. Optical detection devices may be programmed to automatically perform this comparison by means similar to that used by the Quidel Reflective Analyzer, Catalog No. QU0801 (Quidel Corp., San Diego, Calif.). Visual comparison is also possible by visual evaluation of the intensity and a color key. Densitometers and video image analyzers can also be used for this purpose (Immunocytochemistry: A Practical Approach, ed. J. E. Beasely, IRL Press, 1993). As described in U.S. Pat. No. 6,924,153, a video image analyzer uses a digitizing tablet linked to a host computer. The matrix and capture zone are inspected by a microscope or other scanning device and the microscopic image is projected onto the digitizing tablet by a video camera. The computer analyzes the X,Y coordinates of the image to produce a digitized image, which is useful for performing high throughput automated screening of multiple samples.

When a visible dye is used, the signal from the capture zone is directly visually detectable. However, if a fluorescent dye is used the accumulation of the label can be detected by employing a simple fluorescent detection means such as a hand held ultraviolet lamp or a fluorescent microscope. Thus, a variety of detection methods are available to detect the accumulated label on the capture zone.

### Example 10

### Separation of Wave Fronts

This example illustrates how the separation of components on the test strip affects differential arrival of those components through zones of the test strip.

A path of liquid flow was defined along a bibulous substrate from a sample application pad to a mobilization zone to a primary capture zone and then to a secondary capture zone. The primary capture zone contained anti-morphine antibody and the secondary capture zone contained streptavidin. The fluorescent latex particles were placed 20 mm, 13 mm and 4 mm upstream from the primary capture zone. A 2000 ng/ml morphine urine sample was applied to the sample pad, and the fluorescent particles were clearly seen moving behind the solvent front when the fluorescent particles were positioned at 20 mm and 13 mm from the primary capture zone, but not when they were only 4 mm upstream. Since the fluorescent latex particles were coated only with morphine conjugate and not biotin, only the primary capture zone intensity was viewed and measured. A greater distance between the primary capture zone and the position at which the latex particles was applied resulted in a lower signal emanating from the primary capture zone, when the signal was either determined visually or instrumentally.

These results illustrate that a separation distance can be determined for separating label and conjugate on a test strip to maintain migration of the conjugate in advance of the label for preselected distance on the test strip. For example, if migration of conjugate in advance of label is to be maintained at least until conjugate reaches the primary capture zone, then a separation distance is selected that maintains separation of the wave fronts of the label and conjugate until the conjugate reaches the primary capture zone. The particular distance of separation is not fixed, but depends on the characteristics of the label, conjugate and test strip. In particular embodiments in which the label is gold sol coated with streptavidin, the conjugate is BSA-benzoylecognine and BSA-biotin attached to a carrier molecule in optimally determined proportions, and the porous substrate is nitrocellulose (Millipore HF135), the separation distance between the label and the conjugate will vary depending upon the particular mechanism of delayed release employed for the label. For example, if the mechanism of delayed release is to use 3-aminopropyl-trimethoxysilane derivatized glass fiber as the matrix for the sample pad, containing dried impregnated label and conjugate, the separation distance between the label and the conjugate is typically 5-15 mm. Using the other mechanisms of delayed release discussed earlier the distance between label and conjugate will also typically be in the 5-15 mm range.

It will be apparent that the precise details of the methods or compositions described may be varied or modified without departing from the spirit of the described invention. We claim all such modifications and variations that fall within the scope and spirit of the claims below.

1. A test device for detecting an analyte in a liquid sample, the test device comprising:
a bibulous matrix that defines a liquid flow path from a sample receiving zone, through a mobilization zone to a primary capture zone and a secondary capture zone;
a mobilizable conjugate in the mobilization zone;
a mobilizable label upstream of the conjugate;
an immobilized first specific binding partner in the primary capture zone, wherein the first specific binding partner comprises the analyte, a binder for the analyte, or an analog of the analyte;
an immobilized second specific binding partner in the secondary capture zone;
the conjugate comprising a primary specific binding partner for the first specific binding partner in the primary capture zone, and a secondary specific binding partner that binds the label and the second specific binding partner;
wherein application of the liquid sample to the sample receiving zone results in movement of the liquid sample along the liquid flow path to move the label and conjugate distally along the test device, and the label binds the conjugate after the conjugate binds the analyte, the first specific binding partner or the second specific binding partner.

2. The test device of claim 1, wherein the label binds the conjugate after the conjugate binds the analyte, the first specific binding partner or the second specific binding partner.

3. The test device of claim 1, wherein the first specific binding partner in the primary capture zone comprises the analyte or an analog of the analyte.

4. The test device of claim 1, wherein the first specific binding partner in the primary capture zone comprises an antibody that specifically binds the analyte or an analog of the analyte.

5. The test device of claim 1, wherein a ratio of primary specific binding partners to secondary specific binding partners in the conjugate is no more than about 3:1.

6. The test device of claim 1, wherein the primary specific binding partner is an antibody, the analyte or an analog of the analyte.

7. The test device of claim 1, wherein the mobilizable label is in the sample receiving zone.

8. The test device of claim 1, wherein the conjugate does not comprise a colored particle.

9. The test device of claim 1, wherein the label comprises the second specific binding partner.

10. The device of claim 1, wherein the label comprises a colored particulate material.

11. The device of claim 2, wherein the conjugate flows along the liquid flow path in a first wavefront in advance of a second wavefront in which the label flows, at least until the first wavefront reaches the primary capture zone and the conjugate interacts with the first specific binding partner.

12. The device of claim 11, wherein migration of the label along the bibulous matrix in the second wavefront is retarded by one or more of a combination of label size, label weight, label location and selective retardation of release of label from the matrix of the label.

13. The device of claim 11, wherein migration of the label is separated from the conjugate on the bibulous matrix by a sufficient distance that the second wavefront that contains the label does not overtake the first wavefront that contains the conjugate until after the first wavefront reaches the primary capture zone, thereby allowing an increased reaction time between the conjugate and primary capture zone prior to exposure of the primary capture zone to the label.

14. The device of claim 1, wherein the label comprises a labeled biotin binding protein and the second binding partner comprises a biotin binding protein.

15. The device of claim 14, wherein the label comprises a detectable particle.

16. The device of claim 15, wherein the detectable particle comprises colloidal gold, a fluorescent compound, a latex particle, a carbon particle, a dye or an enzyme.

17. The device of claim 1, wherein the primary specific binding partner in the conjugate comprises an antibody that specifically binds the analyte or an analog of the analyte, and the first specific binding partner in the primary capture zone comprises the analyte or analog of the analyte.

18. The device of claim 17, wherein the secondary specific binding partner comprises biotin, and the second specific binding partner in the secondary capture zone comprises a biotin binding protein.

19. The device of claim 18, wherein the immobilized second specific binding partner in the secondary capture zone comprises avidin, streptavidin or a deglycosylated avidin.

20. The device of claim 18, wherein the label comprises the avidin, streptavidin or deglycosylated avidin bound to a detectable particle.

21. The device of claim 1, wherein the primary specific binding partner comprises an antibody that binds the analyte or an analog of the analyte.

22. The device of claim 1, wherein the analyte is follicle stimulating hormone, luteinizing hormone, human chorionic gonadotrophin or a drug.

23. The device of claim 1, wherein the sample is a biological sample.

24. The device of claim 1, wherein the label comprises a biotin binding protein and a detectable particle, the primary binding partner comprises an antibody that specifically binds to the analyte or an analog of the analyte, the secondary binding partner comprises biotin, the first specific binding partner comprises the analyte of an analog of the analyte, and the second binding partner comprises biotin binding protein.

25. The device of claim 1, wherein the label comprises biotin binding protein and a detectable particle, the primary binding partner comprises the analyte of an analog of the analyte, the secondary binding partner comprises biotin, the first specific binding partner comprises an antibody that specifically binds the analyte of an analog of the analyte, and the second specific binding partner comprises biotin binding protein.

26. A method of detecting an analyte in a liquid sample, comprising:
applying the liquid sample to the sample receiving zone of the test device of claim 1, so that the liquid transports the detectable label and the conjugate to the primary capture zone and the secondary capture zone,
wherein the detectable label migrates behind the conjugate to the primary and secondary capture zones, to label the conjugate after it has bound in either the primary or secondary capture zone.

27. The method of claim 26, wherein the first specific binding partner comprises the analyte or an analog of the analyte, the primary specific binding partner comprises an antibody that specifically binds the analyte or the analog of the analyte, the secondary specific binding partner specifically binds the label, and the second specific binding partner comprises the label to which the secondary specific binding partner specifically binds.

28. The method of claim 27, wherein the first specific binding partner comprises an antibody that specifically binds the analyte or an analog of the analyte, the primary specific binding partner comprises the analyte or an analog of the analyte, the secondary specific binding partner specifically binds the label, and the second specific binding partner comprises the label to which the secondary specific binding partner specifically binds.

29. The device of claim 2, wherein the test device is a lateral flow test strip, and the label migrates at a slower rate than the conjugate, such that flow of a liquid solution through the lateral flow matrix results in movement of the labeled first conjugate member and the multivalent composition such that the multivalent composition arrives at the first capture zone sufficiently ahead of the labeled first conjugate member that the multivalent composition binds to the first capture zone before substantial binding of the first conjugate member to the multivalent composition.

30. The device of claim 1, wherein the immobilized specific binding pair member of the first capture zone is the analyte.

31. The device of claim 1, wherein the immobilized first conjugate member of the second capture zone is the immobilized first conjugate member.

32. The device of claim 1, wherein the movement of the labeled first conjugate member behind the multivalent composition produces a separated first wavefront of the labeled first conjugate member and second wavefront of the multivalent composition.

33. The device of claim 1, wherein analyte in the sample binds to the second conjugate member to occupy the specific binding pair member of the second conjugate member such that binding of the second conjugate member to the secondary capture zone is increased, and binding of the labeled first conjugate member to the second conjugate member is substantially delayed until the second conjugate member interacts with the immobilized specific binding pair member in the primary capture zone.

34. The device of claim 1, wherein the label comprises colloidal gold, and enzyme or a fluorescent compound.
